(19) United States
(12) Patent Application Publication
(10) Pub. No.: US 2016/0076728 A1
(43) Pub. Date: Mar. 17, 2016

(54) LIGHTING DEVICE AND A CLEANING CONTROL METHOD OF THE LIGHTING DEVICE

(71) Applicant: OSRAM GmbH, München (DE)

(72) Inventors: Yaojun Feng, Shenzhen (CN); Canbang Yang, Shenzhen (CN); Nimchung Ku, Shenzhen (CN); Yuanyuan He, Shenzhen (CN)

(21) Appl. No.: 14/784,982

(22) PCT Filed: Mar. 17, 2014
(86) PCT No.: PCT/EP2014/055240

§ 371 Date: Oct. 16, 2015

(30) Foreign Application Priority Data
Apr. 17, 2013 (CN) 201310133242.3

(51) Int. Cl. F21V 3/04; H05B 37/04; A61L 9/20; F21V 23/04

(52) U.S. Cl. CPC F21V 3/0472; F21V 23/0464; H05B 37/04; A61L 9/205

(57) ABSTRACT

Various embodiments may relate to a lighting device including a first lighting unit for emitting a first illuminating light, a cover, and a photosensitive coating configured to clean the cover. The lighting device further includes a control device which controls the photosensitive coating to enter an operating state on the basis of whether a situation of ambient environment reaches a first preset condition, and/or which controls the photosensitive coating to enter a non-operating state on the basis of whether the situation of the ambient environment reaches a second preset condition. Furthermore, various embodiments also relate to a cleaning control method of a lighting device.

### RELATED APPLICATIONS

The present application is a national stage entry according to 35 U.S.C. §371 of PCT application No. PCT/EP2014/055240 filed on Mar. 17, 2014, which claims priority from Chinese application No.: 201310133242.3 filed on Apr. 17, 2013, and is incorporated herein by reference in its entirety.

### TECHNICAL FIELD

Various embodiments relate to a lighting device and a cleaning control method of the lighting device.

### BACKGROUND

Along with the development of LED lighting technology, people more and more apply LED lighting devices as light sources to various workaday or commercial environments, for example daily used bulbs, single broadsides as well as stage lighting etc. And thus, people have increasing requirements on the lighting devices using LED lamp technology, for example, requirements on the mechanical performance and electrical performance thereof, particularly requirements on industrial protection, e.g. waterproof and dustproof. To satisfy the requirements on industrial protection of the lighting devices, the related art uses various modes and different materials to seal the lighting device.

However, as the degree sealing has been improved, cleaning the casing and cover of the lighting device when necessary becomes another subject of improving the lighting device. Generally, the user may clean the casing or cover of the lighting device in manually operation modes, for example wiping or flushing. But people need an automatic mode instead of manually operation to clean the casing of the lighting device.

It is suggested according to a solution of the related art that, in order for automatic cleaning of the casing of the lighting device, the casing is treated specially. The object of automatic cleaning can be achieved by applying a layer of special chemicals such as titanium oxide on the external surface of the casing. Said casing can realize the cleaning function according to the irradiation situation of sunlight. However, the casing of such alighting device does not have a self-cleaning function in cases without sunlight continuously, hence, such a lighting device cannot always maintain a clean state of the casing steadily.

### SUMMARY

Various embodiments provide a novel lighting device. Such a lighting device provides the user with a more humanized and simple mode of cleaning the cover, such that the lighting device can realize cleaning of the cover without hand operation. In addition, the lighting device according to various embodiments can realize cleaning of the cover without sunlight, in such way the continuity and stability of maintaining the lighting device clean are realized, and no extra power consumption is necessary for the cleaning in the circumstance with sunlight.

A lighting device according to various embodiments includes a first lighting unit for emitting a first illuminating light, a cover, and cleaning means for cleaning the cover, and the lighting device further includes a control device which controls the cleaning means to enter an operating state on the basis of whether a situation of ambient environment reaches a first preset condition, and/or which controls the cleaning means to enter a non-operating state on the basis of whether the situation of the ambient environment reaches a second preset condition. Since an additional control device is arranged in the lighting device, the lighting device is capable of actively responding according to a preset condition, so as to realize the probability of automatic cleaning. In such way, the cleaning of the lighting device is realized without hand regulation or control.

In various embodiments, whether the situation of the ambient environment reaches the first preset condition is represented by the following condition: whether a first time duration in which a light intensity of a second light in the ambient environment is lower than a first intensity threshold reaches a first time threshold. Therefore, when the detected light is lower than the requirement and the duration of such a circumstance reaches a threshold, the lighting device is provided with a probability of actively responding at this moment; that is, activating the cleaning means to enter the operating state in need of cleaning.

In various embodiments, whether the situation of the ambient environment reaches the second preset condition is represented by the following condition: whether a second time duration in which a light intensity of a second light in the ambient environment is higher than a second intensity threshold reaches a second time threshold. Therefore, when the light required to be detected is higher than the requirement and the duration of such a circumstance reaches a threshold, the lighting device is provided with a probability of actively responding at this moment; that is, activating the cleaning means to enter the non-operating state without the need of cleaning.

In various embodiments, the first time threshold is larger than the second time threshold. By such arrangement, it can be avoided that the cleaning means continue cleaning for an excessively long time, and implementing the cleaning in a suitable shorter period saves the power consumption of the lighting device.

In various embodiments, the control device includes signal providing means and control execution means, wherein the signal providing means provide the light intensity of the second light and the first time duration/second time duration; the control execution means controls the cleaning means to enter the operating state or the non-operating state on the basis of the light intensity of the second light in the ambient environment and the first time duration/second time duration. As an information source of light intensity and time duration, the signal providing means provide the control execution means with basis for judgment, such that the control execution means may decide whether or not the cleaning means work based on the light intensity and time duration of the light intensity.

In various embodiments, the signal providing means include first collecting means providing the light intensity of the second light in the ambient environment as well as a first time unit and a second time unit providing the first time duration and second time duration, respectively. By collecting the irradiation state of the light received by the lighting device and the time duration of the irradiation, the signal providing means can provide the control execution means with basis for controlling the cleaning means, such that the cleaning means can be controllably switched on/off according to the actual conditions.

In various embodiments, the first collecting means is a photo detector. The photo detector detects the irradiation state of the light received by the lighting device and provides the control execution means with a control signal, so as to realize the probability of controlling the cleaning means.

In various embodiments, the photo detector is configured as an ultraviolet detector, and the second light is ultraviolet ray.

The photo detector is implemented to have the function of detecting ultraviolet such that it can be judged directly by the detected result, whether the cover needs to be cleaned.

In various embodiments, the first time unit and the second time unit are respectively a first timer and a second timer. As a simple and effective time counting tool, the timer can auxiliarily provide the control mechanism with basis for judgment, so as to realize the probability of continuously maintaining the cover of the lighting device clean.

In various embodiments, the control execution means include a controller and an actuator connected to each other, wherein the controller generates a control signal according to a parameter provided by the signal providing means and the actuator enters an operating or non-operating state according to the control signal. The functions for sending control instructions and the functions for execution are separated, such that the actuator may react according to a control command of the controller, thereby realizing the controllability.

In various embodiments, the actuator is a second light emitting unit. As a light source, the actuator provides from interior of the lighting device alight ray enabling the cover to self-clean, in such way the cover of the lighting device can react by means of the light ray, such that the object of maintaining the cover clean without hand operation and control is achieved.

In various embodiments, the light emitted by the second light emitting unit is high-frequency light. The cleaning means provided outside the cover reacts to the high-frequency light, so as to function for self-cleaning of the cover.

In various embodiments, the second light emitting unit is an ultraviolet lamp. The cleaning means on the cover can react chemically in response to the ultraviolet ray to perform potential conversion of foul or organic substances attached on the cover so as to clean the cover.

In various embodiments, the first intensity threshold can be different from the second intensity threshold and can be some value selected from ranges from 0.1 to 3.0 m W/cm². In such way, the lighting device can judge based on a preset intensity threshold, whether it is necessary to clean the cover.

In various embodiments, the cleaning means are a coating. Coating coated outside the cover of the lighting device does not affect the appearance of the lighting device and can react actively to the light emitted by the ultraviolet lamp located in the lighting device so as to function for cleaning the cover.

In various embodiments, the coating is a titanium oxide coating. The coating having titanium oxide can react actively to the ultraviolet ray and react chemically with the ultraviolet ray as a catalyst, in such way, in cases without sunshine, a ultraviolet ray is provided in the lighting device such that the cover of the lighting device can be cleaned.

In various embodiments, the coating has thickness of from 10 nm to 10 μm. Such a coating utilizes saved materials to treat the cover of the lighting device while not affecting the appearance of the lighting device, so that the cover has a self-cleaning function in response to the ultraviolet ray.

In various embodiments, the coating is deposited on the external surface of the cover by PVD or CVD technology.

Various embodiments also relate to a cleaning control method of a lighting device including the following steps:

a) detecting a light intensity of a second light in an ambient environment and determining whether a first time duration in which the light intensity of the second light in the ambient environment is lower than a first intensity threshold reaches a first time threshold;

b) if it is, controlling a second light emitting unit to be switched on to provide a supplementary second light;

c) detecting a light intensity of a second light in an ambient environment and determining whether a second time duration in which the light intensity of the second light in the ambient environment is higher than a second intensity threshold reaches a second time threshold;

d) if it is, controlling a second light emitting unit to be switched off to stop providing a supplementary second light.

In the cleaning control method of the lighting device, the sequences of all the steps are interchangeable, and the steps comprised by the control method does not mean that said method only comprises said steps.

In various embodiments, in step a), the first time duration is acquired by time counting by means of a first timer, and in step b), the second time duration is acquired by time counting by means of a second timer. By counts of the first and second timers, the state of ultraviolet irradiation is counted, and it can serve as a basis for controlling the ultraviolet lamp to be switched on/off, so as to realize the continuity of maintaining the cover clean.

In various embodiments, instep d), the first timer is controlled to be reset and the second timer is controlled to be reset. After the first and second timers are reset, it is ready for the next time counting cycle; in such way, the ultraviolet lamp can be controlled to be switched on/off repeatedly in different cases.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the disclosed embodiments. In the following description, various embodiments described with reference to the following drawings, in which:

FIG. 1 is a schematic diagram of a lighting device of one embodiment according to the present disclosure;

FIG. 2 is an operating flow chart of opening of actuator according to one embodiment of the present disclosure; and

FIG. 3 is an operating flow chart of closing of actuator according to one embodiment of the present disclosure.

### DETAILED DESCRIPTION

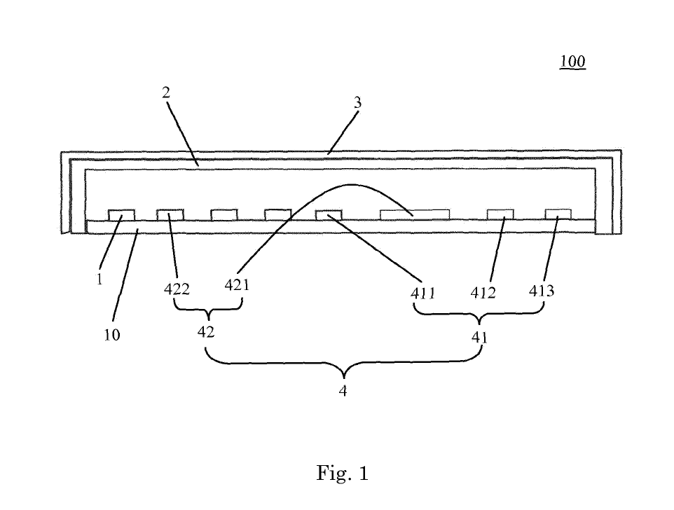

FIG. 1 shows a schematic diagram of a lighting device 100 of one embodiment according to the present disclosure. As shown in FIG. 1, the lighting device of one embodiment according to the present disclosure includes a first lighting unit 1 provided on a carrier 10, a control device 4 as well as a cover 2 for receiving and sealing a first lighting unit 1 and the control device 4. First lighting unit 1 is the light source for providing the illumination function, such as white LED. The cover 2 is provided on its external surface with cleaning means 3 configured as a photosensitive coating, for example, in said embodiment it is a coating having e.g. titanium oxide. Titanium oxide may chemically react with the organic substances and fouling objects attached on the external surface of the cover to produce carbon dioxide and water. In such way, a cleaning function is realized by consuming titanium oxide. It should be pointed out that the cleaning means may also be a chemical coating sensitive to light other than ultraviolet light or other means activated by light. In said embodiment, the coating is applied to the external surface of the cover 2 by means of PVD or CVD technology, and the coating can be configured to have a thickness of from 10 nm to 10 μm, such that it is capable of entering an operating state or a non-operating state according to a control command of the control device 4, thereby realizing the controllability of cleaning of the lighting device 100.

The control device 4 is suggested according to the present disclosure, considering the case that the external environment may not be capable of providing the condition for activating the cleaning means, it is ensured that such activating condition can be complementarily provided even if the external environment cannot satisfy the condition for activating or initiating the cleaning means. Therefore, the control device 4 of the present disclosure further includes signal providing means 41 and control execution means 42. The control device 4 can controls the cleaning means 3 to enter an operating state on the basis of whether the situation of ambient environment reaches a first preset condition, and/or the control device 4 controls the cleaning means 3 to enter a non-operating state on the basis of whether the situation of the ambient environment reaches a second preset condition. In this embodiment, as to the situation of the ambient environment, it is generally considered whether the condition for activating the titanium oxide coating is satisfied. And the activating condition for a chemical coating is generally represented in two aspects, one is the light intensity and the other is irradiation duration of the light ray. Thus, in this embodiment, the signal providing means 41 provide a light intensity D of a second light in the ambient environment and the first time duration/second time duration; the control execution means 42 controls the cleaning means 3 to enter the operating state or the non-operating state on the basis of the light intensity D of the second light in the ambient environment and the first time duration/second time duration.

in order to acquire the light intensity D and the first time duration/second time duration, the signal providing means 41 further include first collecting means 411, a first time unit 412 and a second time unit 413. The first collecting means 411 is configured as an ultraviolet detector having ultraviolet detection function, the first and second time units 412, 413 are configured as first and second timers. It should be pointed out that, as an ultraviolet detector, the first collecting means 411 not only detect ultraviolet ray incident to the cover from the external environment through the cover, but also detect the complementarily provided ultraviolet ray from an actuator 422 of the control execution means 42 (to be described in detail in the following). The ultraviolet detector e.g. can adopt solutions in the prior art, for example, including a photodiode and a UV-filter provided on the photodiode, wherein the UV-filter only allows UV-light to transmit.

The signal providing means 41 provide the control execution means 42 with basis for control so as to realize the function of automatically switch on/off the ultraviolet lamp. In this embodiment, as shown in FIG. 1, the control execution means 42 include a controller 421 and the actuator 422. The controller 421 and actuator 422 are connected to each other, and the controller 421 generates a control signal according to information provided by the first collecting means 411, first time unit 412 and second time unit 413, and the actuator 422 enters an operating or non-operating state according to the control signal. The controller 421 can also control the first time unit 412 and second time unit 413 to be reset.

In this embodiment, the actuator 422 is configured as a second light emitting unit independent of a first light emitting unit and the second light emitting unit is configured as an ultraviolet lamp, for example UVLED, a high-frequency light ray such as ultraviolet light is transmitted by means of the ultraviolet lamp, in such way the effect of activating the coating coated on the external surface of the cover can be realized.

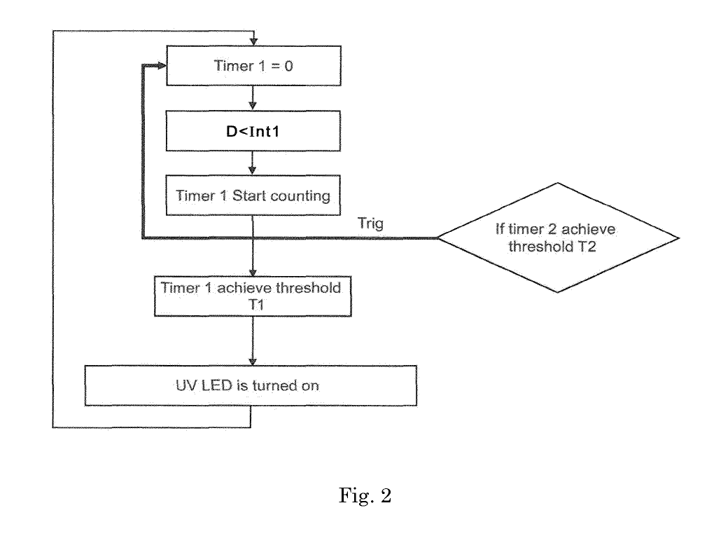

FIG. 2 is an operating flowchart of opening of actuator according to one embodiment of the present disclosure. It can be judged based on a first preset condition whether the ultraviolet lamp should be switched on, i.e. when the light intensity D of the second light in the ambient environment is lower than the first intensity threshold Int1, wherein the first intensity threshold Intl is configured to range from 0.1 to 3.0 mW/cm², the first timer begins to count the time, and when the first timer acquired a first time duration and the first time duration reaches a first time threshold T1, a signal is provided to the control device 4 to control the ultraviolet lamp to be switched on, such that the coating can function for self cleaning under the ultraviolet irradiation.

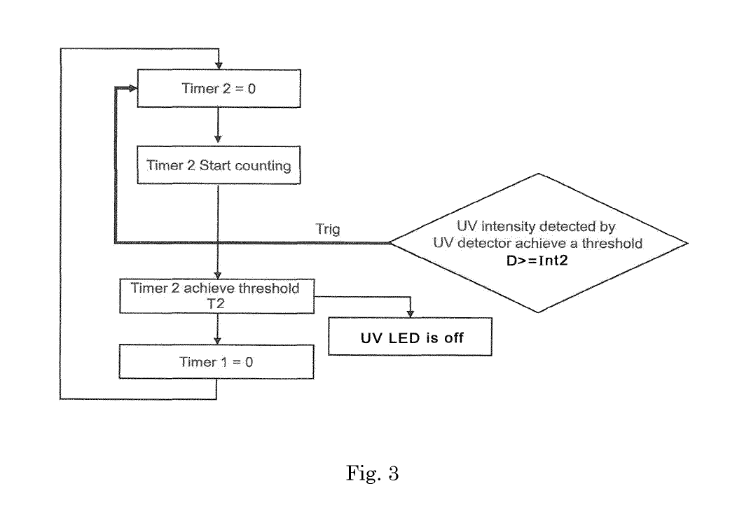

FIG. 3 is an operating flowchart of closing of actuator according to one embodiment of the present disclosure. It can be judged based on a second preset condition whether the ultraviolet lamp should be switched off, i.e. when the light intensity D of the second light in the ambient environment is greater than or equal to a second intensity threshold Int2, wherein the second intensity threshold Int2 can be configured different from the first intensity threshold Int1, the second timer begins to count the time, and when the second timer acquires a second time duration and the second time duration reaches a second time threshold T2, a signal is provided to the controller 421, such that the first timer (as shown in FIG. 2) and the second timer are reset, and the ultraviolet lamp is controlled to be switched off, so as to stop providing the complementary ultraviolet ray, such that the cleaning of the cover 2 of the lighting device 100 is stopped.

The whole working process of the lighting device 100 of the present disclosure is the following:

The first light emitting unit provides the illumination function. The second light emitting unit configured as UVLED providing the cleaning trigger function, is usually off, and here the counts of both first timer and second timer are zero. For example on a cloudy day, the first collecting means 411 can only collect ultraviolet light permeating in the cover from the outside, here the ultraviolet ray may be insufficient. When the light intensity signal received by the controller 421 provided from the first collecting means 411 is lower than the first intensity threshold Intl, the first timer begins to count the time, and when the light intensity signal acquired by the first timer is lower than the first intensity threshold Intl for a first time duration and the first time duration reaches the first time threshold T1, a signal is provided to the controller 421, the controller 421 controls the ultraviolet lamp to be switched on and the count of the first timer is zero. After the ultraviolet lamp is switched on, the first collecting means 411 collect the ultraviolet light permeating in the cover from the outside and the ultraviolet light complementarily provided by the ultraviolet lamp. Since the weather may switch fine, there may be the case that the sum of the ultraviolet light permeating in the cover and the ultraviolet light complementarily provided by the ultraviolet lamp is too much, thus, the controller has also to further decide whether the light intensity D of the second light is greater than or equal to the second intensity threshold Int2. When the light intensity D of the second light in the ambient environment is greater than or equal to the second intensity threshold Int2, the second timer begins to count the time, and when the second timer acquires that the light intensity D of the second light is greater than or equal to the second intensity threshold Int2 for a second time duration and the second time duration reaches the second time threshold T2, a signal is provided to the controller 421, such that the second timer is reset and the ultraviolet lamp is controlled to be switched off.

While the disclosed embodiments have been particularly shown and described with reference to specific embodiments, it should be understood by those skilled in the art that various changes in form and detail may be made therein without departing from the spirit and scope of the disclosed embodiments as defined by the appended claims. The scope of the disclosed embodiments is thus indicated by the appended claims and all changes which come within the meaning and range of equivalency of the claims are therefore intended to be embraced.

1-17. (canceled)

18. A cleaning control method of a lighting device, the method comprising:
a) detecting a light intensity of a second light in an ambient environment and determining whether a first time duration in which the light intensity of the second light in the ambient environment is lower than a first intensity threshold reaches a first time threshold;
b) if it is, controlling a second light emitting unit to be switched on to provide a supplementary second light;
c) detecting a light intensity of a second light in an ambient environment and determining whether a second time duration in which the light intensity of the second light in the ambient environment is higher than a second intensity threshold reaches a second time threshold;
d) if it is, controlling a second light emitting unit to be switched off to stop providing a supplementary second light.

19. The method according to claim 18, wherein in a), the first time duration is acquired by time counting by means of a first timer, and in step c), the second time duration is acquired by time counting by means of a second timer.

20. The method according to claim 19, wherein in d), the first timer is controlled to be reset and the second timer is controlled to be reset.

21. A lighting device comprising:
a first lighting unit for emitting a first illuminating light,
a cover, and
a photosensitive coating configured to clean the cover,
wherein the lighting device further comprises a control device which controls the photosensitive coating to enter an operating state on the basis of whether a situation of ambient environment reaches a first preset condition, and/or which controls the photosensitive coating to enter a non-operating state on the basis of whether the situation of the ambient environment reaches a second preset condition.

22. The lighting device according to claim 21,
wherein whether the situation of the ambient environment reaches the first preset condition is represented by the following condition: whether a first time duration in which a light intensity of a second light in the ambient environment is lower than a first intensity threshold reaches a first time threshold.

23. The lighting device according to claim 22,
wherein whether the situation of the ambient environment reaches the second preset condition is represented by the following condition: whether a second time duration in which a light intensity of a second light in the ambient environment is higher than a second intensity threshold reaches a second time threshold.

24. The lighting device according to claim 23,
wherein the first time threshold is larger than the second time threshold.

25. The lighting device according to claim 23,
wherein the control device comprises:
a photo detector configured to provide the light intensity of the second light;
a first time unit configured to provide the first time duration;
a second time unit configured to provide the second time duration;
a controller configured to provide a control signal to an actuator on the basis of the light intensity of the second light and the first time duration and/or the second time duration, and
the actuator configured to activate the operating state of the photosensitive coating or the non-operating state of the photosensitive coating according to the control signal.

26. The lighting device according to claim 25,
wherein the photo detector is configured as an ultraviolet detector, and the second light is ultraviolet ray.

27. The lighting device according to claim 25, wherein the first time unit and the second time unit are respectively a first timer and a second timer.

28. The lighting device according to claim 25,
wherein the actuator is a second light emitting unit.

29. The lighting device according to claim 28,
wherein light emitted by the second light emitting unit is a high-frequency light ray.

30. The lighting device according to claim 29,
wherein the second light emitting unit is an ultraviolet lamp.

31. The lighting device according to claim 21,
wherein the photosensitive coating is a titanium oxide coating.

32. The lighting device according to claim 21,
wherein the photosensitive coating has a thickness of from 10 nm to 10 μm.

33. A lighting device comprising:
a first lighting unit for emitting a first illuminating light,
a cover, and
cleaning means for cleaning the cover,
wherein the lighting device further comprises a control means for controlling the cleaning means to enter an operating state on the basis of whether a situation of ambient environment reaches a first preset condition, and/or which controls the cleaning means to enter a non-operating state on the basis of whether the situation of the ambient environment reaches a second preset condition.

34. The lighting device according to claim 33,
wherein whether the situation of the ambient environment reaches the first preset condition is represented by the following condition: whether a first time duration in which a light intensity of a second light in the ambient environment is lower than a first intensity threshold reaches a first time threshold.

35. The lighting device according to claim 34,
wherein whether the situation of the ambient environment reaches the second preset condition is represented by the following condition: whether a second time duration in which a light intensity of a second light in the ambient environment is higher than a second intensity threshold reaches a second time threshold.

36. The lighting device according to claim 35,
wherein the first time threshold is larger than the second time threshold.

37. The lighting device according to claim 35,
wherein the control means comprises signal providing means and control execution means, wherein the signal providing means provide the light intensity of the second light and the first time duration and/or the second time duration; the control execution means controls the cleaning means to enter the operating state or the non-operating state on the basis of the light intensity of the second light and the first time duration and/or the second time duration.
